(19) United States
(12) Patent Application Publication
(10) Pub. No.: US 2005/0215937 A1
(43) Pub. Date: Sep. 29, 2005

(54) System and method for filtering leukocytes in cardiac surgery

(76) Inventors: Francis G. Spinale, Charleston, SC (US); Joseph J. Sistino, Charleston, SC (US); Jessica R. Garner, Milford, OH (US); John S. Ikonomidis, Mt. Pleasant, SC (US); Robert E. Stroud, Mt. Pleasant, SC (US)

Correspondence Address: NEEDLE & ROSENBERG, P.C., SUITE 1000, 999 PEACHTREE STREET, ATLANTA, GA, 30309-3915, US

(21) Appl. No.: 11/071,874

(22) Filed: Mar. 3, 2005

Related U.S. Application Data
(60) Provisional application No. 60/550,726, filed on Mar. 5, 2004.

(51) Int. Cl. A61M 37/00

(52) U.S. Cl. 604/6.03; 210/323.1

(57) ABSTRACT

According to one embodiment, a leukocyte reducing filter system comprises a delivery line comprising a plurality of branches, at least a plurality of said branches further comprising a leukocyte reducing filter interposed into said branch, and a stopping mechanism for selectively stopping fluid flow through each of said branches.

### CROSS-REFERENCE TO RELATED APPLICATIONS

This application claims the benefit of U.S. Provisional Application No. 60/550,726, filed Mar. 5, 2004. The aforementioned application is herein incorporated by reference in its entirety.

### ACKNOWLEDGEMENTS

This invention was made with government support under Grant No. R01HL 56603 awarded by The National Institutes of Health. The government has certain rights in the invention.

### BACKGROUND OF THE INVENTION

Cardiopulmonary bypass (CPB) continues to be an integral part of many cardiac surgical procedures. The systemic inflammatory response seen in cardiac patients exposed to CPB, however, can lead to intra-operative and post-operative complications. Moreover, cardioplegic arrest (CA) remains a fundamental requirement for a number of cardiac surgical procedures. Changes in myocardial function, however, can occur following CA and reperfusion.

Matrix metalloproteinases (MMPs) are a family of enzymes responsible for degrading the extracellular matrix, including the basement membrane (Crit Rev in Oral Biol and Med, 4(2):197-250 (1993)), a process likely responsible for the development of altered vascular permeability and subsequent tissue edema post cardiopulmonary bypass (CPB) (J Thorac Cardiovasc Surg, 94:225-233 (1987); Circ Res 75:181-189 (1994); and J Biol Chem 261:2810-2813 (1986)) and following CA and reperfusion. MMP synthesis and activity is tightly regulated under normal conditions, degrading extracellular matrix in coordination with matrix synthesis (Crit Rev in Oral Biol and Med, 4(2):197-250 (1993)). However, in disease states, production of this enzyme has been shown to be upregulated (Circ Res 77:863-868 (1995)). Elevated levels of various subtypes of this enzyme family have been observed in cardiovascular disease states associated with tissue structural changes (J Am Coll Cardiol, 32(2):368-372 (1998)). In the post CPB setting, increased degradative activity directed at the extracellular matrix, especially the basement membrane, can alter endothelial geometrical relationships, compromising the endothelial barrier, ultimately leading to tissue edema and organ dysfunction. Data indicates elevation of select MMPs following CPB, including MMP-9 (Ann Thorac Surg, 71:1518-1523 (2001)). Leukocytes are capable of MMP production and release associated with the inflammatory response.

Leukocyte reducing filters (LRF) have been shown to selectively remove activated neutrophils in patients undergoing cardiopulmonary bypass (Perfusion, 10:291-300 (1995) and Can J Anaesth, 44:131-139 (1997)) without further activation caused by the filtration itself (Journal of Laboratory & Clinical Medicine, 135(3):238-246 (2000)). LRF can be integrated into the arterial line of the extracorporeal circuit (ECC) as well as in the blood cardioplegia line. A blood cardioplegia LRF has been shown to effectively lower WBCs while leaving platelets nearly unaffected (Perfusion, 13:205-210 (1998)).

In patients undergoing cardiac surgery requiring CA, MMP-8 and MMP-9 levels have been shown to be increased four-fold and two-fold within from base line within coronary heart circulation when a leukocyte reducing filter is interposed in the cardioplegic delivery line. Myeloperoxidase levels are also increased. Thus the sequestering of neutrophils within the CA circuit causes degranulation and release of MMPs and myeloperoxidase into the coronary circulation of the patient. Thus the standard use of a leukocyte filter integrated into the cardioplegia line leads to a harmful release of damaging MMPs and myeloperoxidase enzymes. This release can lead to detrimental side effects associated with cardiac bypass surgery and cardioplegic arrest. What is needed in the art is a system and method for filtering leukocytes in the cardioplegia circuit and in a whole body CPB circuit that avoids or reduces the release of leukocyte products including MMPs and myeloperoxidase into the patient from sequestered leukocytes. The disclosed system and method for filtering leukocytes in cardiac surgery meets these needs in the art.

### SUMMARY OF THE INVENTION

Provided herein is a cardioplegic delivery system for filtering leukocytes comprising; a cardioplegic delivery line comprising a plurality of branches, at least a plurality of said branches further comprising a leukocyte reducing filter interposed into said branch, and a stopping mechanism for selectively stopping fluid flow through each of said branches. Optionally, the plurality of branches is parallel and rejoins in a common delivery means for delivery to a subject. Optionally, the stopping mechanism is controlled manually or stopping mechanism is controlled by an automated switching means.

Also provided herein is a cardioplegic delivery system for filtering leukocytes comprising; a first leukocyte reducing filter removeably interposed into a cardioplegia line, wherein blood is circulated through said first filter, a second leukocyte reducing filter, wherein, after blood is circulated through said first filter, said first filter is replaced by said second filter which is removeably interposed into said cardioplegia line and blood is then circulated through said second filter. Optionally, the system further comprises replacing the second filter with a third filter and blood is then circulated through said third filter, replacing the third filter with a fourth filter and blood is then circulated through said fourth filter, and/or replacing the fourth filter with a fifth filter and blood is then circulated through said fifth filter. Of course, additional filters may be similarly interposed beyond the fifth filter.

Also provided is a method of filtering blood comprising; interposing a first leukocyte reducing filter into a first branch of a branched cardioplegia delivery line, interposing a second leukocyte reducing filter into a second branch of a branched cardioplegia delivery line, circulating blood through said first filter through said first branch of said cardioplegia delivery line, and, after blood is circulated through said first filter, closing said first branch to prevent flow through the first filter, and, circulating blood through said second filter through said second branch. Optionally, the method further comprises interposing a third leukocyte reducing filter into a third cardioplegia delivery line branch, wherein, after blood is circulated through the second filter, said second branch is closed to prevent flow through the second filter, and then circulating blood through said third filter through said third branch. Optionally, the method further comprises interposing a fourth leukocyte reducing filter into a fourth cardioplegia delivery branch, wherein after blood is circulated through the third filter, said third delivery branch is closed to prevent flow through the third filter, and then circulating blood through said fourth filter through said fourth branch. Optionally, the system comprises interposing a fifth leukocyte reducing filter into a fifth cardioplegia delivery branch wherein, after blood is circulated through the fourth filter, said fourth delivery branch is closed to prevent flow through the fourth filter, and then circulating blood through said fifth filter through said fifth branch. Of course, additional filters may be similarly interposed beyond the fifth filter. Optionally, the branches are parallel and rejoin in a common delivery means for delivery to a subject. Optionally, the stopping mechanism is controlled manually and/or the stopping mechanism is controlled by an automated switching means.

Also provided is a method of filtering intermittent administrations of blood during cardioplegic arrest comprising; filtering a first administration of blood during cardioplegic arrest through a first leukocyte reducing filter interposed in a cardioplegia delivery line, and filtering a second administration of blood during cardioplegic arrest through a second leukocyte reducing filter interposed in a cardioplegia line, wherein blood does not flow through the first filter during the second administration or any subsequent administration of blood. Optionally, the method further comprises a third administration of blood during cardioplegic arrest through a third leukocyte reducing filter interposed in a cardioplegia line, wherein blood does not flow through the first or second filter during the third administration or any subsequent administration of blood. Optionally, the method further comprises a fourth administration of blood during cardioplegic arrest through a fourth leukocyte reducing filter interposed in a cardioplegia line, wherein blood does not flow through the first or second or third filter during the fourth administration or any subsequent administration of blood. Optionally, the method further comprises a fifth administration of blood during cardioplegic arrest through a fifth leukocyte reducing filter interposed in a cardioplegia line, wherein blood does not flow through the first or second or third or fourth filter during the fifth administration or any subsequent administration of blood. Of course, additional filters may be similarly interposed beyond the fifth filter.

Also provided is a whole body leukocyte reducing filter comprising; a delivery line comprising a plurality of branches, at least a plurality of said branches further comprising a leukocyte reducing filter interposed into said branch, a stopping mechanism for selectively stopping fluid flow through each of said branches. Optionally, the plurality of branches is parallel and rejoins in a common delivery means for delivery to a subject. In one aspect, the stopping mechanism is controlled manually. In another aspect, the stopping mechanism is controlled by an automated switching means.

Also provided herein is a whole body cardio bypass system for filtering leukocytes comprising; a first leukocyte reducing filter removeably interposed into a cardiac bypass line, wherein blood is circulated through said first filter, a second leukocyte reducing filter, wherein blood after blood is circulated through said first filter, said first filter is replaced by said second filter which is removeably interposed into said bypass line and blood is then circulated through said second filter. Optionally, the system further comprises replacing the second filter with a third filter and blood is then circulated through said third filter. Optionally, the system further comprises replacing the third filter with a fourth filter and blood is then circulated through said fourth filter. Optionally the system further comprises replacing the fourth filter with a fifth filter and blood is then circulated through said fifth filter. Optionally, the system further comprising replacing the fifth filter with a sixth filter and blood is then circulated through said sixth filter. Of course, additional filters may be similarly interposed beyond the sixth filter.

Also provided herein is a method of filtering blood comprising; interposing a first leukocyte reducing filter into a first branch of a branched whole body cardio by pass delivery line, interposing a second leukocyte reducing filter into a second branch of a branched whole body cardio bypass delivery line, circulating blood through said first filter through said first branch of said delivery line, and, after blood is circulated through said first filter, closing said first branch to prevent flow through the first filter, and, circulating blood through said second filter through said second branch. Optionally, the method further comprises interposing a third leukocyte reducing filter into a third whole body cardio bypass delivery line branch, wherein after blood is circulated through the second filter, said second branch is closed to prevent flow through the second filter, and then circulating blood through said third filter through said third branch. Optionally, the method further comprises interposing a fourth leukocyte reducing filter into a fourth whole body cardio bypass delivery branch, wherein after blood is circulated through the third filter, said third delivery branch is closed to prevent flow through the third filter, and then circulating blood through said fourth filter through said fourth branch. Optionally, the method further comprises interposing a fifth leukocyte reducing filter into a fifth whole body cardio bypass delivery branch wherein after blood is circulated through the fourth filter, said fourth delivery branch is closed to prevent flow through the fourth filter, and then circulating blood through said fifth filter through said fifth branch. Optionally, the method further comprises interposing a sixth leukocyte reducing filter into a sixth whole body cardio bypass delivery branch wherein after blood is circulated through the fifth filter, said fifth delivery branch is closed to prevent flow through the fifth filter, and then circulating blood through said sixth filter through said sixth branch. In one aspect, the branches are parallel and rejoin in a common delivery means for delivery to a subject. In another aspect, the stopping mechanism is controlled manually. In another aspect, the stopping mechanism is controlled by an automated switching means. Of course, additional filters may be similarly interposed beyond the sixth filter.

Also provided herein is a method of filtering intermittent circulations of blood during whole body cardio bypass comprising; filtering a first circulation of blood during whole body cardio bypass through a first leukocyte reducing filter interposed in a whole body cardio bypass delivery line and, filtering a second circulation of blood during whole body cardio bypass through a second leukocyte reducing filter interposed in a whole body cardio bypass delivery line, wherein blood does not flow through the first filter during the second circulation or any subsequent circulation of blood. Optionally, the method further comprises a third circulation of blood during whole body cardio bypass through a third leukocyte reducing filter interposed in a whole body cardio bypass delivery line, wherein blood does not flow through the first or second filter during the third circulation or any subsequent circulation of blood. Optionally, the method further comprises a fourth circulation of blood during whole body cardio bypass through a fourth leukocyte reducing filter interposed in a whole body cardio bypass delivery line, wherein blood does not flow through the first or second or third filter during the fourth circulation or any subsequent circulation of blood. Optionally, the method further comprises a fifth circulation of blood during whole body cardio bypass through a fifth leukocyte reducing filter interposed in a whole body cardio bypass delivery line, wherein blood does not flow through the first or second or third or fourth filter during the fifth circulation or any subsequent circulation of blood. Optionally, the method further comprises a sixth circulation of blood during whole body cardio bypass through a sixth leukocyte reducing filter interposed in a whole body cardio bypass delivery line, wherein blood does not flow through the first or second or third or fourth or fifth filter during the sixth circulation or any subsequent circulation of blood. Of course, additional filters may be similarly interposed beyond the sixth filter.

Also disclosed herein is a cardioplegic delivery system for filtering leukocytes comprising; a cardioplegic delivery line, a leukocyte reducing filter comprising a plurality of leukocyte reducing filtering compartments interposed into said line, a baffling or shunting mechanism for selectively directing blood flow through each of said filter compartments. In one aspect, the leukocyte reducing filter has five leukocyte reducing filtering compartments. Of course, filters may have more or fewer leukocyte reducing filtering compartments.

Also disclosed herein is a whole body cardio bypass system for filtering leukocytes comprising; a whole body cardio bypass delivery line, a leukocyte reducing filter comprising a plurality of leukocyte reducing filtering compartments interposed into said line, a baffling or shunting mechanism for selectively directing blood flow through each of said filter compartments. In one aspect, the leukocyte reducing filter has six leukocyte reducing compartments. Of course, filters may have more or fewer leukocyte reducing filtering compartments.

Also disclosed herein is a method for filtering leukocytes in a cardioplegic delivery line comprising; interposing a leukocyte reducing filter having a plurality of leukocyte reducing filtering compartments into a cardioplegic delivery line, and selectively directing blood flow through each of said filter compartments.

Also disclosed herein is a method for filtering leukocytes in a whole body cardio bypass delivery line comprising; interposing a leukocyte reducing filter having a plurality of leukocyte reducing filtering compartments into a whole body cardio bypass delivery line, and selectively directing blood flow through each of said filter compartments.

### BRIEF DESCRIPTION OF THE DRAWINGS

The accompanying drawings, which are incorporated in and constitute a part of this specification, illustrate several aspects described below.

FIG. 1A shows WBCs were measured by complete blood counts, which showed WBCs increased in both the non-LRF and LRF groups. WBCs increased up to 6 hours post CPB (p<0.05) when compared to baseline before decreasing at 12 hours post CPB.

FIG. 1B shows WBCs measured as a percent change from 30 minutes into rewarming the patient. There is a trend for WBCs to be higher in the non-LRF group compared to the LRF group, though no significant difference was shown.

FIG. 2A shows Systemic arterial plasma levels for ProMMP-2 increased from baseline to 30 minutes into rewarming in the non-LRF group before returning to within normal levels by 6 hours post CPB. ProMMP-2 increased from baseline to 6 hours post CPB before decreasing at 12 hours post CPB in the LRF group. ProMMP-2 was significantly higher (p<0.05) in the LRF group than the non-LRF group at 6 hours post CPB and significantly higher (p<0.05) in the LRF group when compared to baseline at 12 hours post CPB.

FIG. 2B shows ProMMP-2 measured as a percent change from 30 minutes into rewarming the patient. ProMMP-2 increased in both the non-LRF and LRF groups, but to a higher degree in the LRF group. ProMMP-2 is significantly higher (p<0.05) in the LRF group at 6 and 12 hours post CPB when compared to 30 minutes into rewarming.

FIG. 3A shows Systemic arterial plasma levels for proMMP-9 increased significantly (p<0.05) from baseline to 30 minutes into rewarming the patient when compared to the non-LRF group before decreasing continually to 12 hours post CPB.

FIG. 3B shows ProMMP-9 levels measured as a percent change from baseline. ProMMP-9 levels decreased 30% from 30 minutes into rewarming the patient at 6 hours post CPB in the LRF group.

FIG. 4A shows The percent of neutrophils of the total leukocyte count was measured from a CBC differential at baseline and 12 hours post CPB. Though there was no significant difference in the percent of neutrophils at baseline, there was a significant increase (p<0.05) in both groups 12 hours post CPB compared to baseline. There was no significant difference when comparing the non-LRF and LRF groups to one another at 12 hours post CPB.

FIG. 4B shows The percent of bands, immature neutrophils, of the total leukocyte count was measured from a CBC differential at baseline and 12 hours post CPB. There was no significant difference in the percent of bands at baseline, but there was a significant increase (p<0.05) in the LRF group at 12 hours post CPB when compared to baseline. This suggests that neutrophil levels had decreased and were attempting to replenish themselves, resulting in an increased number of immature neutrophils, or bands, in the body.

FIG. 5 shows a schematic of an exemplary leukocyte reducing filter manifold diagram. Referring to FIG. 5, cardioplegic solution and blood from systemic circulation enters the circuit through a common delivery line 1. A cautionary bypass line 3, with a stop cock 2 or other stopping mechanism branches from the common delivery line and rejoins the circuit at a common delivery line 20. After the cautionary bypass line, the common delivery line branches into a plurality of branches 4-8, each with a separate stop cock or stopping mechanism 9-13. Each branch other than the cautionary bypass line 4-8, has an interposed leukocyte reducing filter 14-18. Each branch, 4-8 rejoins the common delivery line 20 along with the cautionary bypass line 3. At a point distal to the rejoining of the branched lines and the cautionary bypass line, a flush line 19, connects to the common delivery line. Finally, one common delivery line, 21 delivers the cardioplegic solution and blood from circulation along with any flush to the patient.

### DETAILED DESCRIPTION

Before the present system, devices, and/or methods are disclosed and described, it is to be understood that the aspects described below are not limited to specific surgical methods or specific administration methods, as such may, of course, vary. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise.

As used throughout, by a “subject” is meant an individual. In one aspect, the subject is a mammal such as a primate or a human. A patient refers to a subject undergoing cardiac surgery or a procedure requiring cardioplegic arrest.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, “blood”, means blood delivered though a cardioplegic delivery line or through a whole body CBP system. As used herein, blood also includes any mixture of blood and cardioplegic solution, medication, or any other fluid or mixture of fluid delivered through a cardioplegic delivery line. Administration as used herein means the delivery of blood, blood and cardioplegic solution mixture, medication, any other fluid or mixture of fluid delivered to a patient. Effective dosages and schedules for administration may be determined empirically, and making such determinations is within the skill in the art. Generally, the administration will vary with the age, condition, sex and extent of the disease in the patient, route or administration, medication types, surgical complications, the procedure performed, and can be determined by one skilled in the art. The administration amount or schedule can be adjusted by the individual physician or other medical technician or professional. Such administration schedules and dosages will often be decided by the attending surgeon within the scope of sound medical judgment.

Provided herein are systems and methods for filtering leukocytes in cardiac surgery. The systemic inflammatory response seen in cardiac patients exposed to CPB can lead to intra-operative and post-operative complications. Activation of platelets, neutrophils, monocytes, endothelial cells, and lymphocytes have been shown to mediate the principal complications of CPB: bleeding, thromboembolism, fluid retention, and organ dysfunction (Edmunds, Extracorporeal perfusion, 1997:225-94). Matrix metalloproteinases (MMPs), proteolytic enzymes, responsible for degrading the extracellular matrix, including the basement membrane (Birkedal-Hansen et al., Crit Rev in Oral Biol and Med 1993: 4(2): 197-250), are of importance to cardiac surgery because they are a factor in the pathological effects related to CPB. Increased release of several subclasses of MMPs have been shown to occur in the post CPB setting (Joffs et al. Ann Thorac Surg 2001: 71:1518-23). In cardioplegic arrest MMP levels and other enzymes are increased in the patient's coronary circulation. Neutrophils are an important source of MMP release during CA and reperfusion. For example, MMP-8, MMP-9 are elevated when neutrophils degranulate in a CA circuit. Moreover, other harmful enzymes are released from degranulated neutrophils such as myeloperoxidase.

Provided herein is a cardioplegic delivery system for filtering leukocytes comprising a cardioplegic delivery line comprising a plurality of branches, at least a plurality of said branches further comprising a leukocyte reducing filter interposed into said branch, a stopping mechanism for selectively stopping fluid flow through each of said branches. A cardioplegic delivery system can also be referred to as a leukocyte reducing filter system. Although the system can filter different leukocyte types, in a preferred aspect, the system filters by way of non-limiting example, neutrophils. One such filter is a blood cardioplegia filter, for example the Pall™ LeukoGuard-BC®. Such a filter can also be modified to filter a lower volume. The cardioplegic delivery line is made of materials common in the art suitable for use as medical or surgical tubing as would be clear to the skilled artesian. The leukocyte filter is interposed into a branch in a proper orientation to allow filtration of the cardioplegic blood or mixture as would be clear one skilled in the art. The stopping mechanism can be any mechanism capable of stopping flow through a branch of the cardioplegic delivery line. In one aspect, the stopping mechanism can be adjusted into an open or closed position. In an open position, fluid can flow through a given branch of the cardioplegic delivery line. In a closed position, fluid is prevented from flowing through a given branch of the cardioplegic delivery line. Branches are generally alternate pathways in the cardioplegia delivery line that branch off of a common line before the interposed filters and may reform back into a common line after the interposed filters. Circulation through the filter as used herein refers to delivery of blood or cardioplegia mixtures through a filter in a direction of flow towards the patient. The circuit volume can be reduced by recalculating primer volume or with the use of volume expanders as would be clear to one skilled in the art.

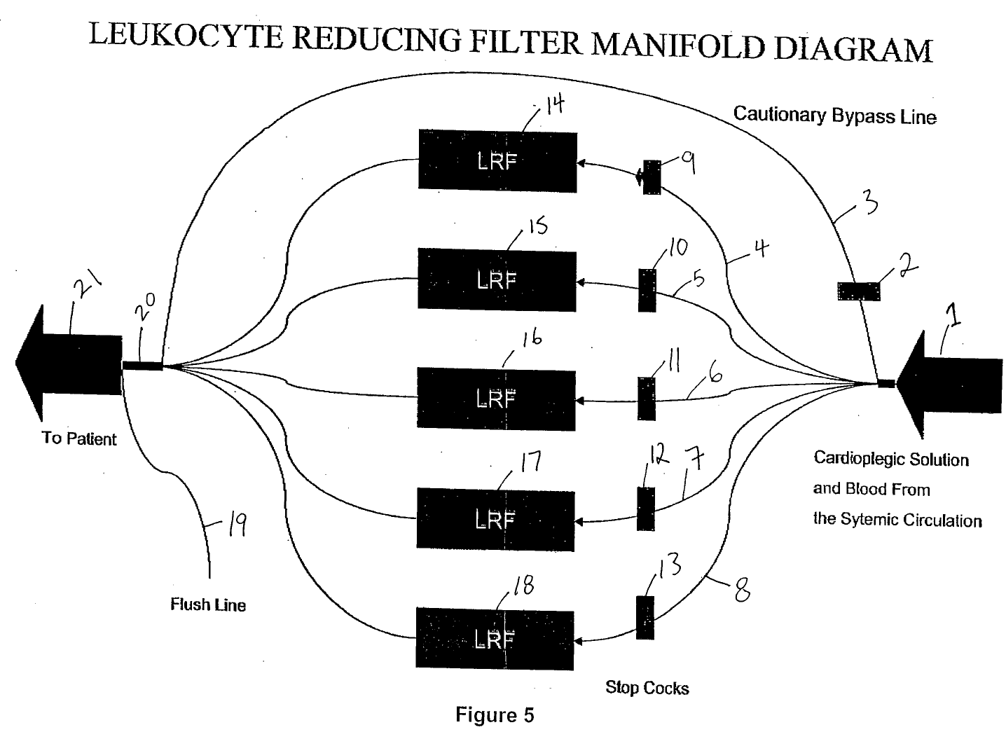

Optionally, the blood is first passed through a branch with an open stopping mechanism and after this first pass the stopping mechanism is closed to prevent flow through this same branch on subsequent administrations. On a second administration of cardioplegic blood, a second stopper can be opened to allow fluid flow through a second branch. One example of a stopping mechanism is a stop cock. Other examples would be any mechanism capable of being opened or closed to allow fluid flow or prevent fluid flow respectively. Specifically, such a mechanism can be, but is not limited to, a clamp or valve. In one aspect, the stopping mechanism can be manually controlled. For example, the surgeon or any other technician or medical professional, can manually open or close the stopping mechanism as desired. In another aspect, the stopping mechanism can be controlled by automatic switching means. For example, software can be programmed or a timer can be used to open or close the stopper at designated times. The timing of opening and closing can depend on the procedure to be performed, the extent of the patient's disease, the physician's preference or other variables as would be clear to one skilled in the art. The plurality of branches can vary depending on the number of filtered administrations to be delivered to a patient. For example, if five administrations of cardioplegic solution or blood are administered to a patient, the cardioplegic delivery line would have five branches with interposed filters or more. By correlating number of branches with number of administrations, each administration can be filtered by a separate filter if desired. Moreover, by filtering through a separate filter each time, the harmful neutrophil and other leukocyte products trapped in the previously used filter are not delivered, or have reduced delivery into the subject's coronary circulation. In one aspect, the plurality of branches is parallel and rejoins in a common delivery means for delivery to the subject. Other aspects of the disclosed cardioplegia delivery system may include flush lines, cardioplegia solution bag, heat exchanger, and pumps. Some of these aspects are illustrated in FIG. 5 and are described in the examples. Other non-illustrated aspects would be clear to one skilled in the art.

Also provided herein, is a cardioplegic delivery system for filtering leukocytes comprising; a first leukocyte reducing filter removeably interposed into a cardioplegia delivery line, wherein blood is circulated through said first filter, a second leukocyte reducing filter, wherein blood after blood is circulated through said first filter, said first filter is replaced by said second filter which is removeably interposed into said cardioplegia delivery line and blood is then circulated through said second filter. For example, a technician or other medical professional can change the leukocyte filter after each administration, prior to a next administration through the same line. Therefore the trapped leukocytes and any harmful products will be reduced or not delivered to the patient when the subsequent administration occurs. In one aspect the system of claim further comprises replacing the second filter with a third filter and blood is then circulated through said third filter. In another aspect, the system further comprises replacing the third filter with a fourth filter and blood is then circulated through said fourth filter. In another aspect, the system further comprising replacing the fourth filter with a fifth filter and blood is then circulated through said fifth filter. Of course, additional filters may be similarly interposed beyond the fifth filter.

Provided herein is a method of filtering blood comprising; interposing a first leukocyte reducing filter into a first branch of a branched cardioplegia delivery line, interposing a second leukocyte reducing filter into a second branch of a branched cardioplegia delivery line, circulating blood through said first filter through said first branch of said cardioplegia delivery line, and, after blood is circulated through said first filter, closing said first branch to prevent flow through the first filter, and, circulating blood through said second filter through said second branch.

The disclosed method removes leukocytes, particularly neutrophils from the cardioplegic mixture administered to a patient during cardioplegic arrest. By removing leukocytes, these cells are not delivered or are reduced in delivery to the patient in the cardioplegic administration. If the same filter is used for each administration during cardioplegia, as is the current practice in the art, neutrophils trapped in the filter degranulate and subsequent administrations through the same filter cause delivery of the degranulation products to the patient. These products include harmful MMPs, including MMP-8 and MMP-9. Myeloperoxidase is also released and delivered to the patient. Therefore, using a single filter, or using no filter both increase harmful neutrophil products and other leukocyte products in a patient undergoing cardioplegia. By using the disclosed method, each administration of blood to a patient during cardioplegia can be passed through and filtered by a separate filter, and the previously used filter with trapped leukocytes can therefore be bypassed. In this way, harmful products from trapped leukocytes are not delivered to the patient or, have a reduced delivery to the patient.

Administration of blood or cardioplegic mixtures including blood, often occur multiple times during cardiac surgery when cardioplegic arrest is used. For example administration of cardioplegic mixtures may occur about every 10, 20, 30, 40 minutes or more. On each administration, in a disclosed embodiment, administered blood passes through an unused leukocyte filter. Overall, the total number of separate administrations of cardioplegic mixtures may occur 2, 3, 4, 5 or more times during a cardiac surgery. One filtration is generally made during the re-warming process. The number and volume of cardioplegic administrations will vary based on a number of factors known to those skilled in the art. Known factors include but are not limited to, patient characteristics, pathological conditions (like hypertrophy of the heart), temperature of the heart muscle, the procedure being performed, and whether antegrade or retrograde administration is used. Both antegrade and retrograde administration is contemplated herein. These and other factors are generally considered by the attending surgeon or other medical professional, within the scope of sound medical judgment.

In one aspect, the method further comprises interposing a third leukocyte reducing filter into a third cardioplegia delivery line branch, wherein after blood is circulated through the second filter, said second branch is closed to prevent flow through the second filter, and then circulating blood through said third filter through said third branch. In another aspect, the method further comprises interposing a fourth leukocyte reducing filter into a fourth cardioplegia delivery branch, wherein after blood is circulated through the third filter, said third delivery branch is closed to prevent flow through the third filter, and then circulating blood through said fourth filter through said fourth branch. In another aspect, the method further comprises interposing a fifth leukocyte reducing filter into a fifth cardioplegia delivery branch wherein after blood is circulated through the fourth filter, said fourth delivery branch is closed to prevent flow through the fourth filter, and then circulating blood through said fifth filter through said fifth branch. Of course, additional filters may be similarly interposed beyond the fifth filter. Optionally, the branches disclosed in the above method are parallel and rejoin in a common delivery means for delivery to a subject. Optionally, the disclosed stopping mechanisms are controlled manually. Optionally, the stopping mechanisms are controlled by an automated switching means.

Also provided herein is a method of filtering intermittent administrations of blood during cardioplegic arrest comprising; filtering a first administration of blood during cardioplegic arrest through a first leukocyte reducing filter interposed in a cardioplegia delivery line and, filtering a second administration of blood during cardioplegic arrest through a second leukocyte reducing filter interposed in a cardioplegia line, wherein blood does not flow through the first filter during the second administration or any subsequent administration of blood. In one aspect, the method further comprises a third administration of blood during cardioplegic arrest through a third leukocyte reducing filter interposed in a cardioplegia line, wherein blood does not flow through the first or second filter during the third administration or any subsequent administration of blood.

In another aspect, the method further comprises a fourth administration of blood during cardioplegic arrest through a fourth leukocyte reducing filter interposed in a cardioplegia line, wherein blood does not flow through the first or second or third filter during the fourth administration or any subsequent administration of blood. In another aspect, the method further comprising a fifth administration of blood during cardioplegic arrest through a fifth leukocyte reducing filter interposed in a cardioplegia line, wherein blood does not flow through the first or second or third or fourth filter during the fifth administration or any subsequent administration of blood. Of course, additional filters may be similarly interposed beyond the fifth filter.

Also provided is a whole body leukocyte reducing filtering system comprising; a delivery line comprising a plurality of branches, at least a plurality of said branches further comprising a leukocyte reducing filter interposed into said branch, a stopping mechanism for selectively stopping fluid flow through each of said branches. Thus the system can be used as a whole body leukocyte reduction filtering system. As used herein, whole body cardiac bypass is intended to include “cardiac bypass line,” “whole body line,” or “cardiobypass delivery line.” These terms refer to a line used in circulating blood through a cardiopulmonary bypass circuit and are also referred to herein as a delivery line and delivery lines. The leukocyte reducing filter can be interposed in the arterial line distal to the bifurcation of the blood cardioplegia line and proximal to the standard arterial line. The leukocyte reducing filter can also be interposed in other locations in the circuit as would be evident to the skilled artesian to achieve the desired result. One such example of a leukocyte reducing filter is the Pall™ LeukoGuard-6. The filter can also be modified to filter a smaller volume. The whole body leukocyte reducing filtering system can be used at critical points following surgery as determined by the attending surgeon or other medical professional. The system is generally used at the time of re-warming or re-waking of the patient's cardiovascular system. For example, the system can be used every 10 minutes for 50 to 60 minutes during reperfusion and re-warming. Other protocols for use of the system during reperfusion and re-warming could be determined by one skilled in the art. Optionally, the plurality of branches is parallel and rejoins in a common delivery means for delivery to a subject. In one aspect, the stopping mechanism is controlled manually. In another aspect, the stopping mechanism is controlled by an automated switching means.

Also provided herein is a whole body cardio bypass system for filtering leukocytes comprising; a first leukocyte reducing filter removeably interposed into a cardiac bypass line, wherein blood is circulated through said first filter, a second leukocyte reducing filter, wherein blood after blood is circulated through said first filter, said first filter is replaced by said second filter which is removeably interposed into said bypass line and blood is then circulated through said second filter. The leukocyte reducing filter can be interposed in the arterial line distal to the bifurcation of the blood cardioplegia line and proximal to the standard arterial line. The leukocyte reducing filter can also be interposed in other locations in the circuit as would be evident to the skilled artesian to achieve the desired result. The whole body leukocyte reducing filtering system can be used at critical points following surgery as determined by the attending surgeon or other medical professional. The system is generally used at the time of re-warming or re-waking of the patient's cardiovascular system. For example, the system can be used every 10 minutes for 50 to 60 minutes during reperfusion and re-warming. Other protocols for use of the system during reperfusion and re-warming could be determined by one skilled in the art. Optionally, the system further comprises replacing the second filter with a third filter and blood is then circulated through said third filter. Optionally, the system further comprises replacing the third filter with a fourth filter and blood is then circulated through said fourth filter. Optionally the system further comprises replacing the fourth filter with a fifth filter and blood is then circulated through said fifth filter. Optionally, the system further comprising replacing the fifth filter with a sixth filter and blood is then circulated through said fifth filter. Of course, additional filters may be similarly interposed beyond the sixth filter.

Also provided herein is a method of filtering blood comprising; interposing a first leukocyte reducing filter into a first branch of a branched whole body cardio bypass delivery line, interposing a second leukocyte reducing filter into a second branch of a branched whole body cardio bypass delivery line, circulating blood through said first filter through said first branch of said delivery line, and, after blood is circulated through said first filter, closing said first branch to prevent flow through the first filter, and, circulating blood through said second filter through said second branch. The leukocyte reducing filter can be interposed in the arterial line distal to the bifurcation of the blood cardioplegia line and proximal to the standard arterial line. The leukocyte reducing filter can also be interposed in other locations in the circuit as would be evident to the skilled artesian to achieve the desired result. The whole body leukocyte reducing filtering system can be used at critical points following surgery as determined by the attending surgeon or other medical professional. The system is generally used at the time of re-warming or re-waking of the patient's cardiovascular system. For example, the system can be used every 10 minutes for 50 to 60 minutes during reperfusion and re-warming. Other protocols for use of the system during reperfusion and re-warming could be determined by one skilled in the art. Optionally, the method further comprises interposing a third leukocyte reducing filter into a third whole body cardio bypass delivery line branch, wherein after blood is circulated through the second filter, said second branch is closed to prevent flow through the second filter, and then circulating blood through said third filter through said third branch. Optionally, the method further comprises interposing a fourth leukocyte reducing filter into a fourth whole body cardio bypass delivery branch, wherein after blood is circulated through the third filter, said third delivery branch is closed to prevent flow through the third filter, and then circulating blood through said fourth filter through said fourth branch. Optionally, the method further comprises interposing a fifth leukocyte reducing filter into a fifth whole body cardio bypass delivery branch wherein after blood is circulated through the fourth filter, said fourth delivery branch is closed to prevent flow through the fourth filter, and then circulating blood through said fifth filter through said fifth branch. Optionally, the method further comprises interposing a sixth leukocyte reducing filter into a sixth whole body cardio bypass delivery branch wherein after blood is circulated through the fifth filter, said fifth delivery branch is closed to prevent flow through the fifth filter, and then circulating blood through said sixth filter through said sixth branch. Of course, additional filters may be similarly interposed beyond the sixth filter. In one aspect, the branches are parallel and rejoin in a common delivery means for delivery to a subject. In another aspect, the stopping mechanism is controlled manually. In another aspect, the stopping mechanism is controlled by an automated switching means.

Also provided herein is a method of filtering intermittent circulations of blood during whole body cardio bypass comprising; filtering a first circulation of blood during whole body cardio bypass through a first leukocyte reducing filter interposed in a whole body cardio bypass delivery line and, filtering a second circulation of blood during whole body cardio bypass through a second leukocyte reducing filter interposed in a whole body cardio bypass delivery line, wherein blood does not flow through the first filter during the second circulation or any subsequent circulation of blood. The leukocyte reducing filter can be interposed in the arterial line distal to the bifurcation of the blood cardioplegia line and proximal to the standard arterial line. The leukocyte reducing filter can also be interposed in other locations in the circuit as would be evident to the skilled artesian to achieve the desired result. The whole body leukocyte reducing filtering system can be used at critical points following surgery as determined by the attending surgeon or other medical professional. The system is generally used at the time of re-warming or re-waking of the patient's cardiovascular system. For example, the system can be used every 10 minutes for 50 to 60 minutes during reperfusion and re-warming. Other protocols for use of the system during reperfusion and re-warming could be determined by one skilled in the art. Optionally, the method further comprises a third circulation of blood during whole body cardio bypass through a third leukocyte reducing filter interposed in a whole body cardio bypass delivery line, wherein blood does not flow through the first or second filter during the third circulation or any subsequent circulation of blood. Optionally, the method further comprises a fourth circulation of blood during whole body cardio bypass through a fourth leukocyte reducing filter interposed in a whole body cardio bypass delivery line, wherein blood does not flow through the first or second or third filter during the fourth circulation or any subsequent circulation of blood. Optionally, the method further comprises a fifth circulation of blood during whole body cardio bypass through a fifth leukocyte reducing filter interposed in a whole body cardio bypass delivery line, wherein blood does not flow through the first or second or third or fourth filter during the fifth circulation or any subsequent circulation of blood. Optionally, the method further comprises a sixth circulation of blood during whole body cardio bypass through a sixth leukocyte reducing filter interposed in a whole body cardio bypass delivery line, wherein blood does not flow through the first or second or third or fourth or fifth filter during the sixth circulation or any subsequent circulation of blood. Of course, additional filters may be similarly interposed beyond the sixth filter.

Also provided herein is a cardioplegic delivery system for filtering leukocytes comprising; a cardioplegic delivery line, a leukocyte reducing filter comprising a plurality of leukocyte reducing filtering compartments interposed into said line, selectively directing blood flow through each of said filter compartments.

In one aspect, the leukocyte reducing filter has 5 leukocyte reducing filtering compartments. In other embodiments, the system could have 2, 3, 4, or more filters. The leukocyte reducing filter comprising a plurality of leukocyte reducing filtering compartments can be modified from a Pall™ LeukoGuard-BC filter. This filter can be modified such that each compartment is separate in that blood to be filtered can be shunted though each component independently of the other compartments. This selective direction can be provided by a system of baffling or shunting mechanisms, which can be controlled manually or automatically to direct blood through each compartment. Therefore, the original filer is functionally divided into a plurality of separate functional filtering units. In this way, the release of sequestered neutrophils and other leukocytes is prevented or reduced because blood can be shunted around the already used compartment of the filter into an unused filter component.

Also provided herein is a whole body cardio bypass system for filtering leukocytes comprising; a whole body cardio bypass delivery line, a leukocyte reducing filter comprising a plurality of leukocyte reducing filtering compartments interposed into said line, a baffling or shunting mechanism for selectively directing blood flow through each of said filter compartments. In one aspect, the leukocyte reducing filter has 6 leukocyte reducing compartments. In other embodiments, the system could have 2, 3, 4, 5, or more filters

Also disclosed herein is a method for filtering leukocytes in a cardioplegic delivery line comprising; interposing a leukocyte reducing filter having a plurality of leukocyte reducing filtering compartments into a cardioplegic delivery line, using a baffling or shunting mechanism for selectively directing blood flow through each of said filter compartments.

Also disclosed herein is a method for filtering leukocytes in a whole body cardio bypass delivery line comprising; interposing a leukocyte reducing filter having a plurality of leukocyte reducing filtering compartments into a whole body cardio bypass delivery line, using a baffling or shunting mechanism for selectively directing blood flow through each of said filter compartments.

### EXAMPLES

Although the present process has been described with reference to specific details of certain embodiments thereof, it is not intended that such details should be regarded as limitations upon the scope of the invention except as and to the extent that they are included in the accompanying claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### Example 1

The Effect of Leukocyte Reduction on Matrix Metalloproteinase Release in Cardiopulmonary Bypass

Patient Selection and Description

Patients (n=17; 15 male, 2 female) undergoing elective coronary artery revascularization with cardiopulmonary bypass (CPB) were entered into this study after obtaining informed consent. This protocol was reviewed and approved by the Institutional Review Board of the Medical University of South Carolina. Patients were randomly assigned to either having two leukocyte reducing filters (LeukoGuard-6 and LeukoGuard-BC, Pall Corporation, East Hills, N.Y., USA) introduced to the CPB circuit or simply receiving conventional therapy with a 40 micron arterial filter (REF AF1040G, Jostra-Bentley, Irvine, Calif., USA). For those patients randomized to the LRF group, one filter (Pall LeukoGuard-6) was placed in the arterial line distal to the bifurcation of the blood cardioplegia line and proximal to the standard arterial line filter. A second filter (Pall LeukoGuard-BC) blood cardioplegia filter was placed in the cardioplegia line distal the roller pump and proximal to the cardioplegia heat exchanger. Both filters were primed according to the manufacturers recommendations. All patients utilized an open reservoir system (BMR-4500, Baxter Healthcare Corporation, Deerfield, Ill., USA) with a standard roller pump (Sarns 8000 and 9000, Ann Arbor, Mich., USA). Blood flow and line pressure were maintained according to hospital protocol for all patients. Patient profiles are presented in Table 1.

TABLE 1
Patient Profiles

| | |
|---|---|
| Height (cm) | 174 ± 10 |
| Weight (kg) | 95 ± 16 |
| Body Surface Area (m2) | 2.1 ± 0.2 |
| Age (years) | 59 ± 9 |
| Graft Number | 3 ± 1 |
| Duration of CPB (min) | 91 ± 21 |
| Duration of Cross Clamp (min) | 70 ± 19 |

n = 17 patients

Sample Collection

Standard induction and maintenance of anesthesia was accomplished with a combination of sufentanil, midazolam, and isoflurane. Systemic heparinization was achieved with a heparin dose of 400 units/kg. CPB was maintained at a cardiac index of 2.0 to 2.4 L/min/m². Initial cardioplegic arrest was accomplished with antegrade administration of a 500 mL solution of D₅ 0.2% sodium chloride (NaCl) containing 29 mL of Tris-hydroxy-methyl-amino methane (THAM) buffer, 34 mL adenosine citrate phosphate dextrose and 60 mEq of potassium chloride (KCl) (120 mEq/L of K⁺). This was followed immediately by retrograde administration of 300 mL of the same cardioplegic solution. Approximately every twenty minutes, cardioplegic arrest was maintained with 400-700 mL retrograde administration of a cardioplegic solution with a lower potassium concentration, 60 mEq/L of K⁺. At the termination of CPB, heparin was neutralized with protamine at a 1:1 ratio. Three milliliters of blood were obtained for matrix metalloproteinase (MMP) levels at baseline (prior to CPB), 30 minutes into rewarming of the patient, 30 minutes post CPB, 6 hours post CPB, and 12 hours post CPB. All samples were placed in EDTA vacutainers, centrifuged, and plasma was stored at −70° C. until assay. In addition, three milliliters of blood were obtained at the above time points for hemodynamic measurements. Complete blood counts (CBC) with differentials were run for baseline and 12 hours post CPB samples. From the CBC with a differential, neutrophil and band (immature neutrophil) levels were measured. CBCs without differentials were run for samples obtained at 30 minutes into rewarming the patient, 30 minutes post CPB, and 6 hours post CPB.

MMP Plasma Assays

This study focused upon one known class of MMPs, gelatinases, which include MMP-2 and MMP-9. Plasma samples were allowed to thaw on ice. Quantification of respective MMP species was done utilizing an enzyme linked immunosorbant assay (ELISA) kit (Amersham Pharmacia Biotech, Buckinghamshire, England). The antisera used for MMP-2 reacts against the proform of MMP-2 (proMMP-2) and does not react against the active form. For MMP-9, the antisera detects the proform of the enzyme (proMMP-9). The ELISA procedure was similar for each MMP, using a two-site assay. Plasma was added to precoated wells containing antibody to the MMP of interest and incubated at 20-30° C. for one hour. The ELISA plate was washed and incubated in the primary MMP antisera conjugated to horseradish peroxidase (25° C., 1 hour). After several washes, tetramethylbenzadine (TMB)/hydrogen peroxidase was added to the mixture and the reaction was allowed to proceed for 30 minutes. The ELISA plate was immediately read at a wavelength of 450nm (Labsystems Multiskan MCC/340, Helsinki, Finland). The concentration of plasma MMP species was determined using known MMP concentrations to generate a standard curve with each set of samples.

Data Analysis

The resultant MMP concentrations were evaluated using analysis of variance (ANOVA) for repeated measures, followed by a Bonferroni corrected t-test where appropriate. Values were then computed as a mean percent change from the 30 minute rewarming timepoint to account for patient variability. All statistical procedures were performed using the BMDP statistical software package (BMDP Statistical Software Inc., Los Angeles, Calif.).

### Results

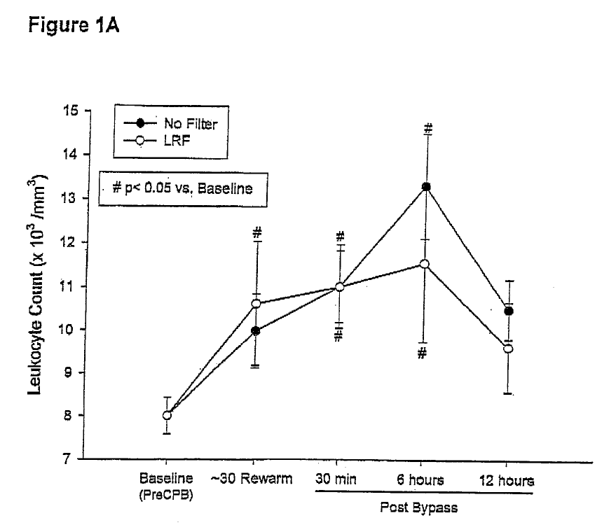

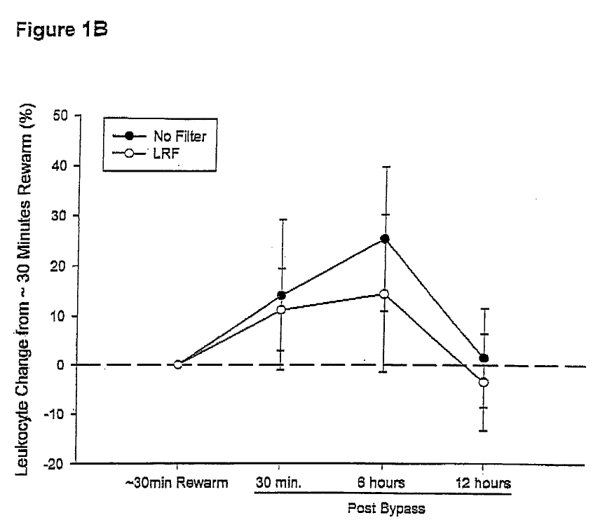

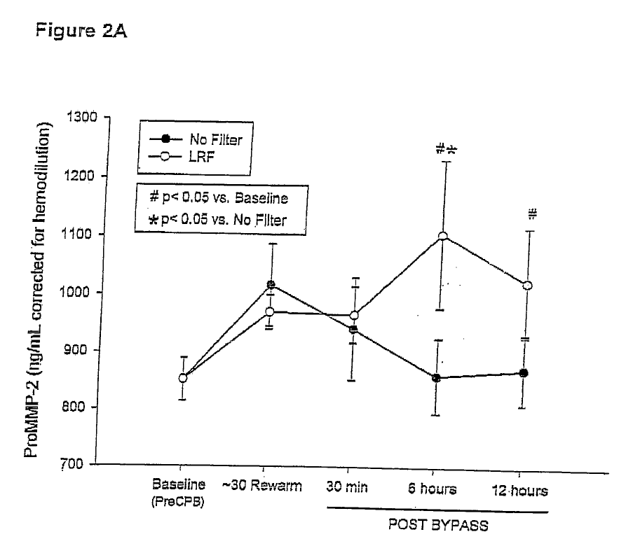

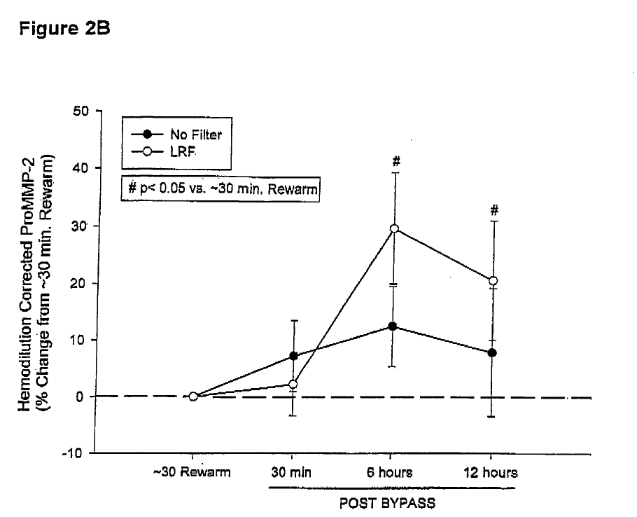

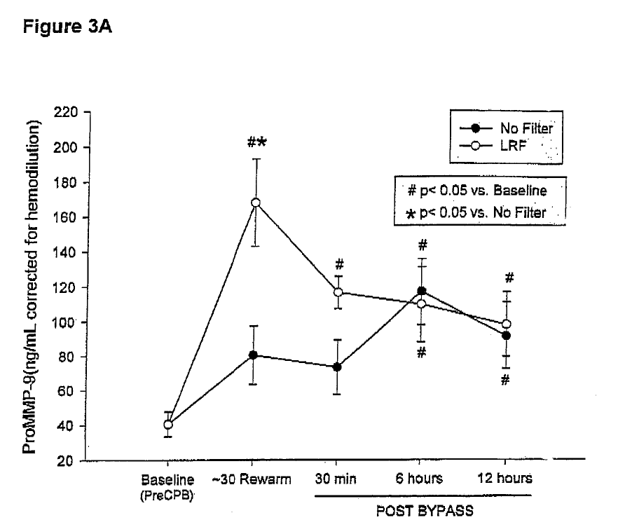

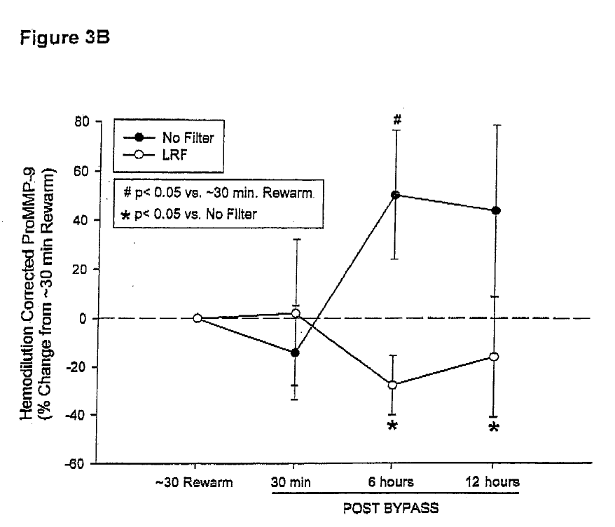

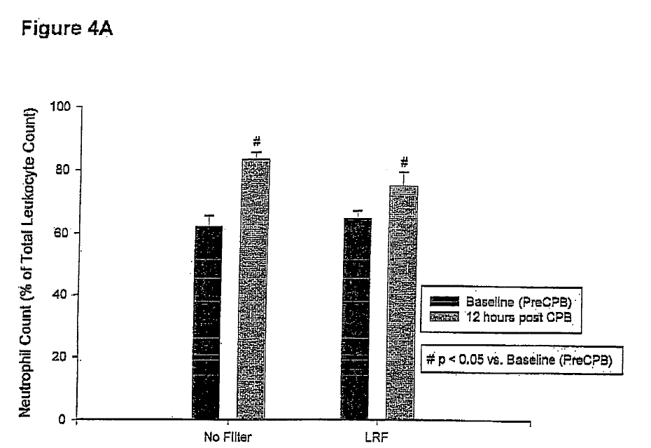

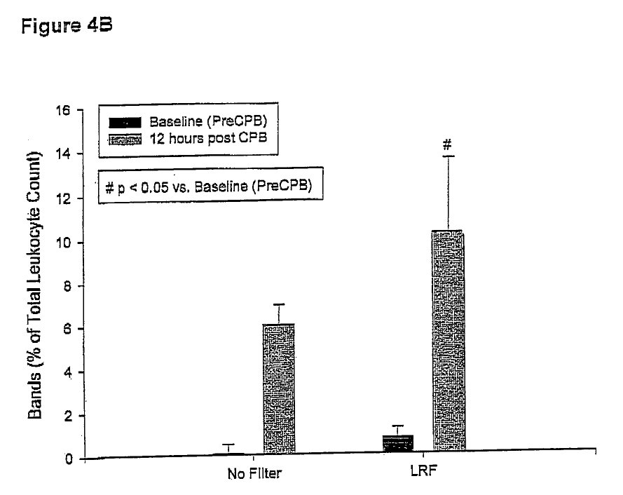

White blood cells (WBCs) increased up to 6 hours post CPB (p<0.05) when compared to baseline before decreasing at 12 hours post CPB (FIG. 1A). WBCs, measured as a percent change from 30 minutes into rewarming the patient, showed a trend for WBCs to be higher in the non-LRF group compared to the LRF group, but did not reach statistical significance (FIG. 1B). Systemic arterial plasma levels for proMMP-2 trended upwards in the non-LRF group, but fell to within normal levels by 6 hours post CPB (FIG. 2A). In contrast, proMMP-2 increased from baseline to 6 and 12 hours post CPB in the LRF group. A similar trend was noted when proMMP-2 was computed as a percent change from the 30 minute rewarming timepoint (FIG. 2B). Systemic arterial plasma levels for proMMP-9 increased significantly (p<0.05) from baseline at the 30 minute rewarming timepoint in the LRF group (FIG. 3A). ProMMP-9 levels were computed as a percent change from the 30 minute rewarming timepoint to account for patient variability in each group and asses the potential impact of the leukocyte reducing filters. When proMMP-9 values were computed as a change from the 30 minute rewarming timepoint, a significant 30% reduction in relative MMP-9 levels was observed (FIG. 3B). ProMMP-9 levels increased 50% from 30 minutes into rewarming at 6 hours post CPB in the non-LRF group. The percent of neutrophils of the total leukocyte count was measured from a CBC differential at baseline and 12 hours post CPB. Though there was no significant difference in the percent of neutrophils at baseline, there was a significant increase (p<0.05) in both groups 12 hours post CPB compared to baseline (FIG. 4A). The percent of bands, immature neutrophils, of the total leukocyte count was measured from a CBC differential at baseline and 12 hours post CPB. There was no significant difference in the percent of bands at baseline, but there was a significant increase (p<0.05) in the LRF group at 12 hours post CPB when compared to baseline (FIG. 4B).

To further investigate the effects of the leukocyte reducing filters, neutrophils and bands, immature neutrophils, were measured as a percent of the total WBC count at baseline and 12 hours post CPB. There was no significant difference in either the neutrophils or band levels at baseline. The percent of neutrophils increased significantly in both the non-LRF and LRF groups at 12 hours post CPB whereas the percent of bands increased significantly in only the LRF group. The increases in neutrophils and bands did not reach statistical significance when comparing the two groups to one another; however, two important findings can be seen from this information. The percent of neutrophils was elevated above the normal range of 45-70% in both groups at 12 hours post CPB. The 12 hour period from termination of CPB until measurement of neutrophils provided sufficient time for neutrophils removed by the LRF to be replenished by the body. The percent of bands, immature neutrophils, is crucial to the interpretation of hematologic measurements from the CBC differentials. The percent of bands was significantly higher from baseline in only the LRF group. This shows that an increased percent of the total WBC count was that of immature neutrophils in the LRF group, but not in the non-LRF group.

### Example 2

Myocardial Specific Release of Matrix Metalloproteinases in Patients Following Cardioplegic Arrest

In eight patients undergoing elective coronary revascularization requiring cardioplegic arrest (CA), MMPs associated with neutrophils (MMP-8 and MMP-9, ng/mL) were measured in the aortic root and coronary sinus before CA (baseline) and after CA with reperfusion. MMP-8 and MMP-9 increased by nearly two-fold following CA compared to baseline (MMP-8: 10.6±2.0 vs. 5.9±0.8; and MMP-9: 109.0±18.0 vs. 69.2±7.9, respectively both p<0.05). In order to carefully examine the role of neutrophils and myocardial MMP release, a second group of patients (n=9) underwent CA in which a leukocyte reducing filter (LRF; LeukoGuard, Pall Corporation) was interposed in the cardioplegic delivery line. With LRF, coronary sinus release of the neutrophil specific MMP-8 increased four-fold from baseline (40.6±3.0 vs. 10.1 V 1.5; p<0.05) and MMP-9 increased two-fold (267±15.9 vs. 128±16.3; p<0.05). Moreover, MMP-8 and MMP-9 were higher in LRF versus nonLRF patients following CA (p<0.05). Finally, a significant burst of myeloperoxidase occurred from baseline in the LRF group following CA indicating degranulation of the sequestered neutrophils (654±34 vs. 1294±107 ng/mL, p<0.05).

Throughout this application, various publications are referenced. The disclosures of these publications in their entireties are hereby incorporated by reference into this application in order to more fully describe the system and methods described herein.

Various modifications and variations can be made to the system and methods described herein. Other aspects of the systems and methods described herein will be apparent from consideration of the specification and practice of the systems and methods disclosed herein. It is intended that the specification and examples be considered as exemplary.

### REFERENCES

1. Birkedal-Hansen H, Moore W G I, Bodden M K, Windsor L J, Birkedal-Hansen B, DeCarlo A, Engler J A. Matrix metalloproteinases: a review. Crit Rev in Oral Biol and Med 1993; 4(2):197-250.
2. Smith E E J, Naftel D C, Blackstone E H, Kirklin J W. Microvascular permeability after cardiopulmonary bypass. J Thorac Cardiovasc Surg 1987; 94:225-33.
3. Galis Z M, Muszynski M, Sukhova G, Simon-Morrissey E, Unemori E, Lark M, Amento E, Libby P. Cytokine-stimulated human vascular smooth muscle cells synthesize a complement of enzymes required for extracellular matrix digestion. Circ Res 1994; 75:181-189.
4. Herron G, Werb Z, Dwyer K, Banda M. Secretion of metalloproteinases by stimulated capillary endothelial cells. J Biol Chem 261:2810-2813, 1986
5. Dollery C M, McEwan J R, Henney A M. Matrix metalloproteinases and cardiovascular disease. Circ Res 1995; 77:863-868.
6. Kai H, Ikeda H, Yasukawa H, Kai M, Seki Y, Kuwahara F, Ueno T, Sugi K, Imaizumi T. Peripheral blood levels of matrix metalloproteases-2 and -9 are elevated in patients with acute coronary syndromes. J Am Coll Cardiol 1998; 32(2):368-72.
7. Joffs C, Gunasinghe H R, Multani M M, Dorman B H, Kratz J M, Crumbley A J, Crawford F A, Spinale F G. Cardiopulmonary bypass induces the synthesis and release of matrix metalloproteinases. Ann Thorac Surg 2001; 71:1518-23.
8. Thurlow P J, Doolan L, Sharp R, et al. Studies of the effect of Pall leukocyte filters LG6 and AV6 on an in vitro simulated extracorporeal circulatory system. Perfusion 1995; 10:291-300.
9. Palanzo D A, Manley N J, Montesano, et al. Clinical evaluation of the LeukoGuard (LG-6) arterial line filter for routine open-heart surgery. Perfusion 1993; 8:489-496.
10. Edmunds L H Jr. Extracorporeal perfusion. In Edmunds L H Jr., ed. Cardiac surgery in the adult. New York: McGraw-Hill, 1997:255-94.
11. Sukhova G K, Schonbeck U, Rabkin E, Schoen F J, Poole R, Billinghurst R C, Libby P. Evidence for increased collangenolysis by interstitial collangenases-1 and -3 in vulnerable human atheromatous plaques. Circulation 2000; 99:2503-09.
12. Parsons S L, Watson S A, Brown P D, Collins H M, Steele R J C. Matrix metalloproteinases. Brit J Surg 1997; 84:160-6.
13. Streuli C. Extracellular matrix remodeling and cellular differentiation. Curr Opin Cell Biol 1999; 11(5)634-40.
14. Hurst T, Johnson D, Cujec B, et al. Depletion of activated neutrophils by a filter during cardiac valve surgery. Can J Anaesth 1997; 44:131-9.
15. Smit J J, de Vries A J, Gu Y J, van Oeveren W. Filtration of activated granulocytes during cardiopulmonary bypass surgery: a morphologic and immunologic study to characterize the trapped leukocytes. Journal of Laboratory & Clinical Medicine. 2000; 135(3):238-46.
16. Suzuki I, Ogoshi N, Chiba M, Komatsu T, Moizumi Y. Clinical evaluation of a leukodepleting blood cardioplegia filter (BC1B) for elective open-heart surgery. Perfusion 1998; 13:205-10.
17. Di Salvo, C, Louca L L, Pattichis K, Hooper J, Walesby R K. Does activated neutrophil depletion on bypass by leukocyte filtration reduce myocardial damage? A preliminary report. J Cardiovascular Surgery 1996; 37(6 Suppl 1)93-100.

1. A leukocyte reducing filter system comprising;
a delivery line comprising a plurality of branches,
at least a plurality of said branches further comprising a leukocyte reducing filter interposed into said branch,
a stopping mechanism for selectively stopping fluid flow through each of said branches.

2. The system of claim 1, wherein the plurality of branches is parallel and rejoins in a common delivery means for delivery to a subject.

3. The system of claim 1, wherein the delivery line is a cardioplegic delivery line.

4. The system of claim 1, wherein the delivery line is a cardiac bypass line.

5. The system of claim 1, wherein the delivery line comprises five branches.

6. The system of claim 5, wherein a leukocyte reducing filter is interposed into each of the five branches.

7. The system of claim 1, wherein the stopping mechanism is controlled manually.

8. The system of claim 1, wherein the stopping mechanism is controlled by an automated switching means.

9. A leukocyte reducing filter system comprising;
a first leukocyte reducing filter removeably interposed into a delivery line, wherein blood is circulated through said first filter,
a second leukocyte reducing filter, wherein after blood is circulated through said first filter, said first filter is replaced by said second filter which is removeably interposed into said delivery line and blood is then circulated through said second filter.

10. The system of claim 9, wherein the delivery line is a cardioplegic delivery line.

11. The system of claim 9, wherein the delivery line is a cardiac bypass line.

12. A leukocyte reducing filter system comprising;
a delivery line,
a leukocyte reducing filter comprising a plurality of leukocyte reducing filtering compartments interposed into said line,
a baffling or shunting mechanism for selectively directing blood flow through each of said filter compartments.

13. The system of claim 12, wherein the delivery line is a cardioplegic delivery line.

14. The system of claim 12, wherein the delivery line is a cardiac bypass line.

15. A method of filtering blood comprising;
interposing a first leukocyte reducing filter into a first branch of a branched delivery line,
interposing a second leukocyte reducing filter into a second branch of a branched delivery line,
circulating blood through said first filter through said first branch of said delivery line, and, after blood is circulated through said first filter, closing said first branch to prevent flow through the first filter, and,
circulating blood through said second filter through said second branch.

16. The method of claim 15, wherein the branches are parallel and rejoin in a common delivery means for delivery to a subject.

17. The method of claim 15, wherein the delivery line is a cardioplegic delivery line.

18. The method of claim 15, wherein the delivery line is a cardiac bypass line.

19. The method of claim 15, wherein the delivery line comprises five branches.

20. The method of claim 19, wherein a leukocyte reducing filter is interposed into each of the five branches.
